(19) United States
(12) Patent Application Publication
(10) Pub. No.: US 2018/0280258 A1
(43) Pub. Date: Oct. 4, 2018

(54) COMPOSITION COMPRISING A SOFT-FOCUS FILLER AND A COMPOSITE PIGMENT BASED ON NON-SPHERICAL ALUMINA AND ON A METAL OXIDE

(71) Applicant: L'OREAL, Paris (FR)

(72) Inventors: Olfa BCHIR, CHEVILLY LARUE (FR); Gaetan CHEVALIER, CHEVILLY LARUE (FR); Ludovic THEVENET, CHEVILLY LARUE (FR)

(73) Assignee: L'OREAL, Paris (FR)

(21) Appl. No.: 15/765,642

(22) PCT Filed: Sep. 19, 2016
(86) PCT No.: PCT/EP2016/072198

§ 371 Date: Apr. 3, 2018

(30) Foreign Application Priority Data
Oct. 14, 2015 (FR) 1559750
Oct. 14, 2015 (FR) 1559751
Oct. 14, 2015 (FR) 1559752

(51) Int. Cl. A61K 8/02; A61K 8/06; A61K 8/26; A61K 8/29; A61K 8/58; A61Q 1/02

(52) U.S. Cl. CPC A61K 8/0266; A61K 8/06; A61K 8/26; A61K 8/29; A61K 2800/651; A61Q 1/02; A61K 2800/43; A61K 2800/612; A61K 8/585

(57) ABSTRACT

The present invention relates to a composition, in particular comprising a physiologically acceptable medium, in particular for coating keratin materials, more particularly for making up and/or caring for keratin materials, comprising: a) at least one soft-focus filler, and b) at least one composite pigment comprising at least one non-spherical aluminium oxide, at least one metal oxide and at least one surface-treatment agent. According to another subject of the invention, the present invention relates more particularly to a composition as previously defined, in emulsion form. The present invention also relates to a composition comprising: a) at least one soft-focus filler, other than boron nitride and b) at least one composite pigment comprising at least one non-spherical aluminium oxide, at least one metal oxide and at least one surface-treatment agent, and c) boron nitride particles. The invention also relates to a process for coating keratin materials, more particularly for making up and/or caring for keratin materials, such as the skin, characterized in that it comprises the application to the keratin materials of a composition as previously defined.

The present invention relates to a composition, in particular comprising a physiologically acceptable medium, in particular for coating keratin materials, more particularly for making up and/or caring for keratin materials, comprising:

a) at least one soft-focus filler, and
b) at least one composite pigment comprising at least one non-spherical aluminium oxide, at least one metal oxide and at least one surface-treatment agent. More particularly, the composition is in emulsion form.

The present invention also relates to a composition comprising:

a) at least one soft-focus filler, other than boron nitride and
b) at least one composite pigment comprising at least one non-spherical aluminium oxide, at least one metal oxide and at least one surface-treatment agent, and
c) boron nitride particles.

The invention also relates to a process for coating keratin materials, more particularly for making up and/or caring for keratin materials, such as the skin, characterized in that it comprises the application to the keratin materials of a composition as previously defined.

The skin is not a smooth surface of uniform colour, and has reliefs and microreliefs such as pores, wrinkles, fine lines, spots, scars and dry areas, which form a somewhat bumpy surface. Quite often, this surface, with its irregularities, forms a pleasant-looking whole, but the irregularities are such that the surface is occasionally considered unattractive.

Cosmetic makeup and/or care compositions are commonly employed for hiding, smoothing out and/or unifying skin relief imperfections such as pores, wrinkles and/or fine lines and/or scars. In this regard, many solid or fluid, anhydrous or non-anhydrous formulations have been developed to date.

The application of a makeup composition such as a foundation is the most effective approach for enhancing the beauty of irregular skin, by making it possible to hide stains and dyschromias, to reduce the visibility of relief imperfections such as pores and wrinkles, and to conceal spots and acne marks; in this regard, coverage is one of the main properties sought. In the compositions, use is generally made of pigments based on metal oxides such as iron oxides and titanium oxides that can be modified with a surface-treatment agent. However, the latter are opaque, and have a tendency to accumulate in the reliefs such as pores and wrinkles, to create a contrast in terms of opacity and colour and to accentuate these relief imperfections.

In order to reduce this relief imperfection accentuation phenomenon, it has been proposed to combine, with the metal oxide pigments, soft-focus fillers, also called “fillers with a soft-focused effect” or “haze-effect fillers”. However, the compositions containing this combination do not make it possible to completely satisfactorily reduce this accentuation.

In addition to the problem of accentuation, compositions with high metal oxide pigment contents have a tendency to produce a feeling of thickness, and of an unnatural mask on keratin materials such as the skin. Makeup compositions such as foundations containing a lower metal oxide pigment content introduce less opacity and less of a “mask” effect and give the skin a more natural effect. Nevertheless, they do not make it possible to obtain a very good uniformity of the complexion because of their lower opacity.

Furthermore, the foundations and face care compositions commonly used comprise at least one aqueous phase, such as oil-in-water emulsions or water-in-oil emulsions. It is important for the pigments present in these compositions to have good dispersibility with a view to obtaining a stable and uniform composition. It is also important for the compositions containing them to provide cosmetic and in particular sensory properties that are satisfactory for the comfort of the consumer, in particular compositions which are soft, fresh and light on application.

There remains therefore a need to find novel cosmetic compositions based on metal oxide pigments which make it possible to effectively smooth out and conceal skin imperfections, to substantially reduce or even eliminate the effects of accentuation of the reliefs and mask effect on keratin materials such as the skin, and to obtain good uniformity of the complexion and a more natural appearance.

During its research, the applicant has discovered, surprisingly, that this objective can be achieved with a composition comprising:

a) at least one soft-focus filler, and
b) at least one composite pigment comprising at least one non-spherical aluminium oxide, at least one metal oxide and at least one surface-treatment agent.

This discovery forms the basis of the invention.

The present invention relates to a composition, in particular comprising a physiologically acceptable medium, in particular for coating keratin materials, more particularly for making up and/or caring for keratin materials, comprising:

a) at least one soft-focus filler, and
b) at least one composite pigment comprising at least one non-spherical aluminium oxide, at least one metal oxide and at least one surface-treatment agent.

According to another subject of the invention, the present invention relates more particularly to a composition as previously defined, in emulsion form.

The present invention also relates to a composition, in particular comprising a physiologically acceptable medium, in particular for coating keratin materials, more particularly for making up and/or caring for keratin materials, comprising:

a) at least one soft-focus filler, other than boron nitride and
b) at least one composite pigment comprising at least one non-spherical aluminium oxide, at least one metal oxide and at least one surface-treatment agent, and
c) boron nitride particles.

The invention also relates to a process for coating keratin materials, more particularly for making up and/or caring for keratin materials, such as the skin, characterized in that it comprises the application to the keratin materials of a composition as previously defined.

### Definitions

In the context of the present invention, the term “keratin materials” is intended to mean the skin and more particularly the areas of the face, the cheeks, the hands, the body, the legs and thighs, the area around the eyes, and the eyelids.

The term “physiologically acceptable” is intended to mean compatible with the skin and/or appendages thereof, which has a pleasant colour, odour and feel and which does not generate unacceptable discomfort (tingling, tautness), which may dissuade consumers from using this composition.

The term “filler” should be understood to mean colourless or white solid particles of any shape which are in a form that is insoluble and dispersed in the medium of the composition.

In the context of the present invention, the term “soft-focus” denotes a hazy effect which hides the skin's microreliefs. This effect makes it possible in particular to attenuate via an optical effect skin defects such as marks, wrinkles or fine lines.

These soft-focus fillers may also be referred to as fillers with a soft-focus effect or haze-effect fillers.

The term “pigments” is intended to mean white or coloured, mineral or organic particles, which are insoluble in an aqueous medium, and which are intended to colour and/or opacify the resulting composition and/or film. These pigments may be white or coloured, and mineral and/or organic.

The term “composite pigment” is intended to mean any pigment comprising at least two materials of different chemical structure. In the context of the present invention, the composite pigment comprises at least one metal oxide and at least one composite pigment comprising at least one non-spherical aluminium oxide (alumina) which are different from one another and at least one surface-treatment agent.

The term surface-treatment agent is intended to mean any chemical compound capable of blocking, adhering to or attaching to by covalent, physical or ionic bonding, partially or totally, the surface of the metal oxide(s) and/or the non-spherical alumina constituting the composite pigment of the invention.

### Soft-Focus Fillers

As stated previously, the claimed compositions comprise at least one soft-focus filler other than boron nitride and in particular as detailed hereinafter.

Fillers of this type are particularly advantageous in so far as they can make imperfections hazy. As indicated previously, the performance of these fillers is advantageously increased by means of using them in a composition according to the invention.

The haze effect is characterized by Haze and transparency (transmission TH) measurements in a reference white base defined below. The Haze corresponds to the percentage of light scattered relative to the total transmittance according to standard ASTM D 1003 (Standard Test Method for Haze and Luminous Transmittance of Transparent Plastics).

25 μm films of composition are applied onto 50 μm polyethylene (PE) films. The film is then measured after 1 hour of drying at ambient temperature (25° C.). Finally, the film is placed in the machine and transparency and Haze measurements are taken.

The soft-focus filler(s) other than hydrophobic silica aerogels is (are) introduced in an amount of 12% by weight relative to the total weight of the white base described below.

In the particular case where the filler is a hydrophobic silica aerogel, the amount introduced into the white base is 4% by weight relative to the total weight of the white base described below.

| | | % by |
|---|---|---|
| Phase | INCI Name | weight |
| A1 | CETYL PEG/PPG-10/1 DIMETHICONE | 3.89 |
| | POLYGLYCERYL-4 ISOSTEARATE | 1.30 |
| | ETHYLHEXYL METHOXYCINNAMATE | 9.74 |
| | PHENYL TRIMETHICONE | 9.74 |
| | DIMETHICONE (and) PEG/PPG-18/18 | 1.39 |
| | DIMETHICONE | |
| | DIMETHICONE | 6.71 |
| | CYCLOHEXASILOXANE | 5.36 |
| | DIMETHICONE | 12.07 |
| A2 | DIMETHICONE (and) DIMETHICONE/PEG-10/15 | 0.70 |
| | CROSSPOLYMER | |
| | DIMETHICONE (and) | 0.60 |
| | DIMETHICONE/POLYGLYCERIN-3 | |
| | CROSSPOLYMER | |
| B1 | WATER | qs 100 |
| | BUTYLENE GLYCOL | 6.48 |
| | MAGNESIUM SULFATE | 0.89 |
| | PROPANEDIOL | 6.48 |
| | ADENOSINE | 0.05 |
| | SORBITOL | 0.65 |
| | PHENOXYETHANOL | 0.91 |
| C1 | DENAT ALCOHOL | 6.48 |

In particular, the soft-focus filler(s) in the white base is (are) characterized in that the Haze is greater than 60% and the transmission TH is greater than 60%, and more preferentially the Haze is greater than 70% and the transmission TH is greater than 70% according to standard ASTM D 1003 (Standard Test Method for Haze and Luminous Transmittance of Transparent Plastics).

Needless to say, as indicated below, the compositions according to the invention may in parallel also contain additional standard fillers, it being understood that those skilled in the art will take care to ensure that they do not choose fillers whose nature or amount in the composition would impair the soft-focus effect afforded by the soft-focus fillers.

For the purposes of the present invention, the term “fillers” should be understood as meaning colourless or white, mineral or organic, natural or synthetic solid particles of any form, which are in an insoluble and dispersed form in the medium of the composition.

The soft-focus fillers that may be used in the composition according to the invention are in particular characterized by a refractive index of between 1.33 and 2. The refractive index is measured according to a conventional method under normal temperature (25° C.) and atmospheric pressure (760 mmHg) conditions.

They will generally comprise or be constituted of particles with an average size of less than or equal to 25 μm, in particular less than or equal to 20 m and in particular less than or equal to 15 m.

The particle size and the size distributions are measured at 25° C. by static light scattering using a particle size analyser such as the Mastersizer 2000 from Malvern. The light intensity scattered by the particles as a function of the angle at which they are lit is converted to size distribution according to the Mie theory. This theory, which is exact for isotropic particles, also makes it possible to determine, for non-spherical particles, an “effective” particle diameter. This theory is particularly described in the publication by Van de Hulst, H. C., Light Scattering by Small Particles, Chapters 9 and 10, Wiley, New York, 1957. The size of the particles can be determined by the values D[10], D[50] and D[90].

D[10] represents the maximum size that 10% by volume of the particles have.
D[50] represents the maximum size that 50% by volume of the particles have.
D[90] represents the maximum size that 90% by volume of the particles have.

These particles may be of any shape and in particular may be spherical or non-spherical.

In particular, the soft-effect filler is chosen from
polytetrafluoroethylene powders,
polyurethane powders,
carnauba microwaxes,
synthetic wax microwaxes,
silicone resin powders,
hollow hemispherical particles of silicone,
acrylic polymer powders, such as expanded vinylidene/acrylonitrile/methylene methacrylate microspheres, polymethyl methacrylate powders, crosslinked copolymers (in particular crosslinked with ethylene glycol dimethacrylate) of ethylhexyl acrylate and of at least one monomer chosen from acrylic acid, methacrylic acid or an ester thereof, and mixtures thereof;
polyethylene powders, in particular comprising at least one ethylene/acrylic acid copolymer,
crosslinked elastomeric organopolysiloxane powders and crosslinked elastomeric organopolysiloxane powders coated with silicone resin,
starch powders,
polyamide powders,
silica powders such as silica particles surface-treated with a mineral wax, amorphous silica microspheres, silica microbeads, hydrophobic silica aerogel particles,
silicates, in particular alumina silicates,
talc of number-average size less than or equal to 3 microns, silica/TiO₂ composites,
barium sulfate particles,
boron nitride particles,
composite powers of talc/TiO₂/alumina/silica,
spherical cellulose beads,
   and mixtures thereof.

As soft-focus fillers that may be used according to the invention, mention may in particular be made of:
talc/TiO₂/alumina/silica composite powders, for instance those sold, for example, under the name Coverleaf AR-80® by the company Catalyst & Chemicals,
silica/TiO₂ composites, such as those sold by the company Sunjin Chemical under the name Sunsil Tin 50,
acrylic polymer powders, in particular hollow or solid spheres of crosslinked polymethyl (meth)acrylate polymer, for instance the Jurimer MBI® PMMA particles from Nihon Junyoki of average size 8 μm, the hollow spheres of PMMA sold under the name Covabeads LH85® by the company Sensient Cosmetic Technologies, the Ganzpearl GMP0820® PMMA particles from the company Ganz Chemical; the expanded vinylidene/acrylonitrile/methylene methacrylate microspheres sold under the name Expancel®; the crosslinked copolymers of at least one monomer chosen from acrylic acid, methacrylic acid and an ester thereof, and mixtures thereof such as the spheres sold by the company Daito Kasei Kogyo under the name Makibeads SP-10® or those sold by the company Serisui Plastics under the name Techpolymer ACP-8C®;
hollow hemispherical silicone particles, of INCI name Methylsilanol/silicate crosspolymer, as described in applications JP-2003 128 788 and JP-A-2000-191789, for instance NLK 500®, NLK 506® and NLK 510@ from Takemoto Oil and Fat;
hydrophobic silica aerogel particles (INCI name: Silica silylate), such as the product sold by the company Dow Corning under the name Dow Corning VM-2270 Aerogel Fine Particles;
crosslinked elastomeric organopolysiloxane powders, such as Dow Corning 9701 Cosmetic Powder® from the company Dow Corning (INCI name: Dimethicone/vinyl dimethicone crosspolymer); or the product sold by the company Dow Corning under the name EP-9801 Hydrocosmetic Powder (INCI name: Dimethicone/vinyl dimethicone crosspolymer (and) butylene glycol);
crosslinked elastomeric organopolysiloxane powders coated with silicone resin, in particular with silsesquioxane resin, as described, for example, in U.S. Pat. No. 5,538,793. Such elastomer powders are sold under the names KSP-100®, KSP-101@, KSP-102@, KSP-103@, KSP-104® and KSP-105® by the company Shin-Etsu; or the product having the INCI name Vinyl dimethicone/methicone silsesquioxane crosspolymer treated with PEG-7 glyceryl cocoate, Polyquaternium-7 and methylsilanol tri-PEG-8 glyceryl cocoate sold by the company Miyoshi Kasei under the name MW-SRP-100;
starch powders, in particular aluminium starch octenylsuccinate, such as the product sold by the company Akzo Nobel under the name Dry Flo Plus;
polyamide powders, such as Nylon® 12 powder, in particular the product sold under the name Orgasol 2002 Extra D Nat Cos® by the company Atochem;
spherical cellulose beads, such as those sold by the company Daito Kasei under the name Cellulobeads®;
and mixtures thereof.

According to one particular embodiment, the composition according to the invention comprises:

a) at least one soft-focus filler chosen from:
polytetrafluoroethylene powders,
polyurethane powders,
carnauba microwaxes,
synthetic wax microwaxes,
silicone resin powders,
hollow hemispherical particles of silicone,
expanded vinylidene/acrylonitrile/methylene methacrylate microspheres,
copolymers of ethylhexyl acrylate and of at least one monomer chosen from acrylic acid, methacrylic acid and mixtures thereof and also esters thereof,
polyethylene powders, in particular comprising at least one ethylene/acrylic acid copolymer,
crosslinked elastomeric organopolysiloxane powders and crosslinked elastomeric organopolysiloxane powders coated with silicone resin,
starch powders,
silica particles surface-treated with a mineral wax,
hydrophobic silica aerogel particles,
talc of number-average size less than or equal to 3 microns, silica/TiO₂ composites,
barium sulfate particles,
composite powers of talc/TiO₂/alumina/silica,
spherical cellulose beads, and mixtures thereof; and
   b) at least one composite pigment comprising at least one non-spherical aluminium oxide, at least one metal oxide and at least one surface-treatment agent.

More particularly, the composition is in emulsion form.

According to one particular embodiment, the composition according to the invention comprises:

a) at least one soft-focus filler chosen from:
polytetrafluoroethylene powders,
polyurethane powders,
carnauba microwaxes,
synthetic wax microwaxes,
silicone resin powders,
hollow hemispherical particles of silicone,
acrylic polymer powders, such as expanded vinylidene/acrylonitrile/methylene methacrylate microspheres, spherical crosslinked polymethyl methacrylate polymer powders, crosslinked copolymers of at least one monomer chosen from acrylic acid, methacrylic acid and mixtures thereof and also esters thereof,
polyethylene powders, in particular comprising at least one ethylene/acrylic acid copolymer,
crosslinked elastomeric organopolysiloxane powders and crosslinked elastomeric organopolysiloxane powders coated with silicone resin,
starch powders,
silica particles surface-treated with a mineral wax,
hydrophobic silica aerogel particles,
silicates, in particular alumina silicates,
talc of number-average size less than or equal to 3 microns, silica/TiO₂ composites,
barium sulfate particles,
composite powers of talc/TiO₂/alumina/silica,
spherical cellulose beads and mixtures thereof, and
   b) at least one composite pigment comprising at least one non-spherical aluminium oxide, at least one metal oxide and at least one surface-treatment agent, and
   c) boron nitride particles.

According to one particularly preferred embodiment, the soft-focus fillers used according to the invention are chosen from:
spherical powders of crosslinked polymethyl methacrylate polymer (INCI name Methyl Methacrylate Crosspolymer), such as the product sold under the name Covabead LH 85® by the company Sensient Cosmetic Technologies,
crosslinked copolymers of at least one monomer chosen from acrylic acid, methacrylic acid and mixtures thereof and also esters thereof (INCI name: Acrylates/Ethylhexyl Acrylate Crosspolymer) in particular in the form of hollow spheres such as those sold by the company Daito Kasei Kogyo under the name Makibeads SP-10® or those sold by the company Serisui Plastics under the name Techpolymer ACP-8C®, and
mixtures thereof.

According to one even more particularly preferred embodiment, the soft-focus fillers used according to the invention are chosen from crosslinked copolymers of at least one monomer chosen from acrylic acid, methacrylic acid and mixtures thereof and also esters thereof (INCI name: Acrylates/Ethylhexyl Acrylate Crosspolymer) in particular in the form of hollow spheres such as those sold by the company Daito Kasei Kogyo under the name Makibeads SP-10® or those sold by the company Serisui Plastics under the name Techpolymer ACP-8C®.

A composition according to the invention may comprise from 2% to 35% by weight, in particular from 3% to 30% by weight and more particularly from 4% to 25% by weight of soft-focus filler(s) relative to the total weight of said composition.

### Composite Pigments

As previously stated, the compositions of the invention contain at least one composite pigment comprising at least one non-spherical aluminium oxide (or alumina), at least one metal oxide and at least one surface-treatment agent.

The term “non-spherical aluminium oxide” is intended to mean an aluminium oxide in three dimensions (length, width and thickness or height) for which the ratio of the greatest dimension to the smallest dimension is greater than 1.2. The dimensions of the particles of the invention are evaluated by scanning electron microscopy and image analysis. They include particles of parallelepipedal shape (rectangular or square surface area), discoid shape (circular surface area) or ellipsoid shape (oval surface area), characterized by three dimensions: a length, a width and a height. When the shape is circular, the length and the width are identical and correspond to the diameter of a disc, whereas the height corresponds to the thickness of the disc. When the surface is oval, the length and the width correspond, respectively, to the large axis and the small axis of an ellipse and the height corresponds to the thickness of the elliptic disc formed by the platelet. When it is a parallelepiped, the length and the width may be of identical or different dimensions: when they are of the same dimension, the shape of the surface of the parallelepiped is a square; in the contrary case, the shape is rectangular. As regards the height, it corresponds to the thickness of the parallelepiped.

Preferentially, the aluminium oxide or alumina is in planar form or in platelet form. The length and the width are generally greater than the thickness.

The aluminium oxide will generally comprise or be constituted of particles with an average size of less than or equal to 25 μm, in particular less than or equal to 20 μm and in particular less than or equal to 15 m.

Preferentially, the thickness of the aluminium oxide is less than 0.5 μm.

By way of example of alumina particles that can be used in the composite pigments of the invention, mention may be made of those sold under the name Alusion® by the company Advances Nanotechnology Limited.

According to the present invention, the composite pigment comprises at least one metal oxide and at least one aluminium oxide. They can be dry-mixed together and surface-treated in order to block them, to attach them or to adhesively bond them together, i.e. directly or indirectly, each one being in loose powder form. According to one particular embodiment, the metal oxide and the aluminium oxide substrate can each be combined with a liquid such as water so as to form a sludge, a dispersion or a solution, then said sludges, dispersions or solutions can subsequently be mixed together. The resulting liquid mixtures can be filtered so as to form a composite pigment.

The agent for surface-treating the composite pigments of the invention may be hydrophobic or hydrophilic.

The term “hydrophobic surface-treatment agent” is intended to mean any compound capable of rendering the composite pigments hydrophobic or more dispersible in an oily phase.

The term “hydrophilic surface-treatment agent” is intended to mean any compound capable of rendering the composite pigments hydrophilic or more dispersible in an aqueous phase.

The surface of the particles may be bonded, by covalent or ionic bonding, to an organic molecule or to a silicone molecule or may be adsorbed to said treatment agent, or the particle may be physically bonded to a layer of a material. The surface-treatment agent may be attached to the particle by a coupling agent, a bonding group or a functional group (silane, ether, ester, etc.).

The hydrophobic surface-treatment agent may comprise a hydrophobic portion which can be chosen from alkyl, allyl, aryl, vinyl, alkylaryl, organosilicone, diorganosilicone, dimethicone, methicone, polyurethane, silicone-polyurethane, and fluoro or perfluoro derivatives thereof. Among the hydrophobic surface-treatment agents, mention may also be made of lauroyl lysine, isopropyl titanium trisostearate (ITT), ITT and dimethicone crosspolymer, ITT and amino acid, ITT/triethoxycaprylylsilane crosspolymer, waxes (carnauba wax), fatty acids (stearic acid), HDI/trimethylol hexylactone crosspolymer, PEG-8 methyl ether triethoxysilane, aloe, jojoba ester, lecithin, perfluoroalcohol phosphate, and magnesium stearate (MM).

Among these hydrophobic surface-treatment agents, mention may for example be made of aluminium laurate, aluminium stearate, metal soaps such as magnesium myristate; amino acids such as lauroyl lysine, chitin, collagen, fluoro compounds, natural waxes, polyacrylates, silicones such as dimethicone copolyols, dimethicones and methicones; silanes such as triethoxycaprylylsilane (or triethoxyoctylsilane), trimethoxycaprylylsilane; titanates; urethanes, perfluoropolymethylisopropyl ethers, styrene/acrylate copolymers, and mixtures thereof. Preferentially, use will be made of a surface-treatment agent chosen from methicones, silanes, dimethicone copolyols, and mixtures thereof.

The agent for surface treatment of the composite pigments of the invention may be hydrophilic, such as those described for example in Cosmetics & Toiletries, February 1990, Vol. 105, p 53-64. Mention may be made, for example, of amino acids, C₁-C₅ alkanolamines, silicon oxides (silica), sodium hexametaphosphate or glycerol or mixtures thereof, PEG-12 dimethicone, sodium glycerophosphate, PEG-7 glyceryl cocoate+methylsilanol tri-PEG-8 glyceryl cocoate+polyquaternium-7, chitosan, methoxy PEG-10 propyltrimethoxysilane, PEG/PPG-18/18 dimethicone, microcristalline cellulose, and polyethylene glycol alkoxysilanes.

As surface-treatment agent, use will more preferentially be made of an alkylsilane, preferably a C₁-C₂₀-alkylsilane, more preferentially a C₁-C₁₂alkylsilane, and even more particularly a tri(C₁-C₄)alkoxy(C₁-C₁₂)alkylsilane such as triethoxyoctylsilane, also known as triethoxycaprylylsilane (INCI name: Triethoxycaprylylsilane).

The term “alkylsilane” is intended to mean a silicone hydride compound (SiH₄) substituted with at least one alkyl radical and possibly also being substituted with 1 to 3 radicals such as alkoxy radicals.

According to one particular mode, the surface-treatment agent attaches the metal oxide and the non-spherical alumina substrate either directly or indirectly. The metal oxide is attached to said substrate by adhesive forces between the treatment agent and the alumina and between the treatment agent and the metal oxide. In particular, the adhesive forces maintain the metal oxide and the alumina, each being in loose powder form.

The surface-treatment agent is preferably present in a proportion of at least 0.50% by weight, in particular of at least 0.75% by weight and more particularly of at least 1% by weight relative to the weight of the composite pigment; according to one particular mode, in a proportion of less than 30%, in particular of less than 25% by weight, more particularly of less than 20% by weight relative to the weight of the composite pigment.

According to one particular form of the invention, the composite pigment may also additionally comprise silica particles.

The composite pigment comprises the alumina substrate, partially coated with the metal oxide.

According to one particular form of the invention, the alumina substrate in platelet form comprises an upper surface and a lower surface which are separated by a border and the metal oxide randomly covers said surfaces, but the border is substantially free of metal oxide.

The term “border substantially free of metal oxide” signifies that at least 90% of the surface of the border of the non-spherical transparent substrate, in particular the alumina, does not comprise metal oxide attached thereto.

According to one particular form of the invention, 5% to 99% of the surface of the alumina substrate is covered with the metal oxide of the composite pigment.

The metal oxides that can be used in the composite pigments of the invention are preferably chosen from titanium oxides, and yellow, red, brown, amber or black iron oxides.

According to one particular form of the invention, the metal oxide(s) is (are) present in an amount ranging from 25% to 85% and preferably from 30% to 75% by weight relative to the total weight of the composite pigment.

According to one particular form of the invention, the metal oxide(s) is (are) present in an amount ranging from 30% to 75% by weight relative to the total weight of the composite pigment.

According to one particular form, the composite pigment is chosen from

(i) a pigment comprising at least one titanium oxide, a platelet alumina and an alkylsilane, in particular a C₁-C₂₀ alkylsilane, more preferentially a C₁-C₁₂alkylsilane, and even more particularly a tri(C₁-C₄)alkoxy(C₁-C₁₂)alkylsilane such as triethoxyoctylsilane, also known as triethoxycaprylylsilane (INCI name: Triethoxycaprylylsilane);
(ii) a pigment comprising an iron oxide, a platelet alumina and an alkylsilane, in particular a C₁-C₂₀ alkylsilane, more preferentially a C₁-C₁₂alkylsilane, and even more particularly a tri(C₁-C₄)alkoxy(C₁-C₁₂)alkylsilane such as triethoxyoctylsilane, also known as triethoxycaprylylsilane (INCI name: Triethoxycaprylylsilane);
(iii) mixtures thereof.

Among the composite pigments that can be used according to the invention, mention may be made of those sold under the trade name Covalumine® by the company Sensient, such as:
Covalumine Sonoma Red AS® (INCI name: Alumina (and) CI 77491 (and) Triethoxycaprylylsilane)
Covalumine Sonoma Yellow AS® (INCI name: Alumina (and) CI 77492 (and) Triethoxycaprylylsilane)
Covalumine Sonoma Black AS® (INCI name: Alumina (and) CI 77499 (and) Triethoxycaprylylsilane) (and) silica)
Covalumine Atlas White AS® (INCI name: Alumina (and) Titanium Oxide (and) Triethoxycaprylylsilane).

The composite pigments in accordance with the invention can be prepared according to one of the processes described in U.S. Pat. No. 7,964,178.

They can in particular be prepared according to a process comprising the following steps:
a) dry-combining of the metal oxide(s) and the alumina substrate, in particular the alumina at ambient temperature (20-25° C.) and normal pressure (1 atmosphere), so as to form a dry mixture;
b) addition of the surface-treatment agent so as to form a mixture;
c) mixing at ambient temperature (20-25° C.) and normal pressure (1 atmosphere).

According to one particular mode, steps 1 and 2 can be carried out simultaneously. In other words, the metal oxide(s), the alumina substrate and the surface-treatment agent can be combined in the first step and then mixed.

In step 2, the surface-treatment agent can be added in order to substantially attach or adhesively bond the metal oxide(s) and the alumina to one another.

In step 3, the mixture can be stirred for at least 30 seconds, in particular at least 5 minutes and more particularly at least 12 minutes. According to one particular mode, the stirring can last less than 30 minutes, preferably less than 25 minutes, more particularly less than 20 minutes.

According to one particular mode, the metal oxide(s) and/or the surface-treatment agent can be hydrated. Water can also be added to the mixture while dry and/or to the final mixture.

According to one particular mode, the final mixture comprising water can be dried after stirring at ambient temperature and normal pressure. It can also be dried by heating in an oven at a temperature of at least 70° C., preferably of at least 75° C., more particularly of at least 80° C. According to one particular mode, the final mixture can be heated at a temperature of less than 110° C., preferably less than 100° C., in particular less than 95° C., more particularly less than 90° C. The heating time can range from 1 to 2 hours; according to one particular mode, for a period of less than 48 hours, preferably less than 36 hours and more particularly less than 18 hours.

The composite pigment(s) in accordance with the invention is (are) preferably present in concentrations ranging from 0.01% to 80% by weight and more preferentially from 1.5% to 70% by weight relative to the total weight of the composition.

According to one particular form of the invention, the composite pigment/soft-focus filler weight ratio ranges from 0.01 to 4, and preferably from 0.7 to 1.5. According to one particular form, the composition according to the invention comprises:

a) at least one soft-focus filler chosen from acrylic polymer powders, in particular powders of crosslinked polymethyl methacrylate polymer (INCI name: Methyl Methacrylate Crosspolymer), crosslinked copolymers of at least one monomer chosen from acrylic acid, methacrylic acid and mixtures thereof and also esters thereof (INCI name: Acrylates/Ethylhexyl Acrylate Crosspolymer) and mixtures thereof; and
b) at least one composite pigment comprising an iron oxide, a platelet alumina and a tri(C₁-C₄)alkoxy(C₁-C₁₂)alkylsilane, in particular octyltriethoxysilane, and/or a composite pigment comprising a titanium oxide, a platelet alumina and a tri(C₁-C₄)alkoxy(C₁-C₁₂)alkylsilane, in particular triethoxyoctylsilane also known as triethoxycaprylylsilane (INCI name: Triethoxycaprylylsilane, as previously defined.

According to one particular form, the composition according to the invention comprises:

a) at least one soft-focus filler chosen from acrylic polymer powders, in particular powders of crosslinked polymethyl methacrylate polymer (INCI name: Methyl Methacrylate Crosspolymer), crosslinked copolymers of at least one monomer chosen from acrylic acid, methacrylic acid and mixtures thereof and also esters thereof (INCI name: Acrylates/Ethylhexyl Acrylate Crosspolymer) and mixtures thereof; and
b) at least one composite pigment comprising an iron oxide, a platelet alumina and a tri(C₁-C₄)alkoxy(C₁-C₁₂)alkylsilane, in particular octyltriethoxysilane, and/or a composite pigment comprising a titanium oxide, a platelet alumina and a tri(C₁-C₄)alkoxy(C₁-C₁₂)alkylsilane, in particular triethoxyoctylsilane also known as triethoxycaprylylsilane (INCI name: Triethoxycaprylylsilane, as previously defined; c) boron nitride.

### Coverage/Opacity Properties

According to one particular mode, the compositions of the invention have a coverage characterized by a contrast ratio (CR) preferably ranging from 16 to 80 and more preferentially from 20 to 79%.

The coverage is measured according to the following protocol:

### Coverage Measurement

The application may be performed with an automatic spreader.

The coverage (contrast ratio) of the first and second compositions is evaluated by preparing a 25 μm film on a contrast card (such as a Byk Chart PA-2814) having a white part and a black part, using an automatic spreader.

It is left to dry for 1 hour at ambient temperature: (20-25° C.).

Colour measurements were taken using a Minolta CM600d spectrocolorimeter. The colorimeter gives numerical data representing the absolute value and the colour difference between a reference sample and a sample to be controlled. In our study, we use the absolute values of each film sample on the white background for the colour, integrating the black background for the calculation of the contrast ratio.

The operating conditions are as follows:

CIE 1964 10° standard observer
Illuminant CIE type D65

Measuring geometry: Scattered measuring geometry/8° and observation, D65/10°, specular component included (SCI) mode, mean aperture (10 mm) on the white and black backgrounds.

The apparatus is equipped with an optical device that produces scattered light, placed in a spherical cavity lined with a white coat, which induces multiple reflection of the light. An anti-glare trap can remove the glare effects from the surface of the sample.

The spectra are expressed as colorimetric coordinates in the Commission Internationale de l'Eclairage CIELab76 space according to recommendation 15:2004.

The contrast ratio is calculated by taking the arithmetic mean of the Y values on the black background, divided by the mean value of Y on the white background, multiplied by 100.

$$Contrast Ratio = \frac{Mean ( Yblack )}{Mean ( Ywhite )} * 100$$

### Boron Nitride

There are several polymorphic forms of boron nitride: hexagonal form boron nitrides (denoted h-BN), rhombohedral form boron nitrides (denoted r-BN), amorphous form boron nitrides (denoted a-BN), turbostratic boron nitrides (denoted t-BN), cubic form boron nitrides (denoted c-BN) and wurtzite-type hexagonal form boron nitrides (denoted w-BN).

Preferentially, the boron nitride particles in accordance with the invention are chosen from turbostratic boron nitride particles, that is to say the crystallization planes of which may be slightly offset with respect to the theoretical position of crystallization. Turbostratic boron nitride is a precursor of hexagonal form boron nitride (h-BN). It has the same type of characteristics and physical properties as exfoliated hexagonal boron nitride.

Preferentially, the boron nitride particles have an oxygen content ranging from 0.05% to 3% by weight, more preferentially from 0.1% to 2.5% by weight, relative to the total weight of the particle.

Preferentially, the boron nitride particles have an average particle size ranging from 0.1 to 25 μm and preferably from 0.3 to 15 μm.

The particle size is measured according to a method of distribution by laser diffraction with an apparatus of the type Microtrac from Nikkiso or Mastersizer from Malvern, in particular by measuring the D[10], D[50] and D[90] values.

D[10] represents the maximum size that 10% by volume of the particles have.
D[50] represents the maximum size that 50% by volume of the particles have.
D[90] represents the maximum size that 90% by volume of the particles have.

The boron nitride particles can be modified with a surface-treatment agent making it possible to confer thereon amphiphilic properties and to promote the dispersibility thereof in the compositions comprising an oily phase and/or an aqueous phase.

Treatment agents that can be chosen include dimethylpolysiloxanes (dimethicone), linear siloxane polymers end-blocked with trimethoxysiloxy groups, polymethylhydrogenosiloxanes which are linear polysiloxanes called methicones, and polyoxyalkylenated polyalkylethersiloxanes such as the polymer PEG-8 methyl ether dimethicone.

The boron nitride particles in accordance with the invention will be chosen more particularly from the following commercial products:

UHP-1010® from Carborundum,
PUHP 1030L® from the company Saint Gobain Ceramics,
Boron Nitride Powder TRES BN PUHP 3002® from the company Saint Gobain Ceramics,
TRES BN PUHP 30005® from the company Saint Gobain Ceramics,
Leau3002® (INCI name: Boron Nitride (and) PEG-8 Methyl Ether Dimethicone) from the company Saint Gobain,
Ronaflair Boroneige SF-3 117774® from the company Merck,
Ronaflair Boroneige SQ-6® from MERCK,
Softtouch Boron Nitride CC 6059® from the company Momentive,
Softtouch Boron Nitride CC 6097® from the company Momentive,
Softtouch CCS102J® from the company Momentive.

The boron nitride particles in accordance with the invention are preferably used in concentrations ranging from 0.1% to 20% by weight and preferentially from 0.5% to 15% by weight relative to the weight of the composition.

### Cosmetic Compositions

According to one particular mode, the compositions of the invention comprise a physiologically acceptable medium.

The term “physiologically acceptable” is intended to mean compatible with the skin and/or appendages thereof, which has a pleasant colour, odour and feel and which does not generate unacceptable discomfort (tingling, tautness, redness), which may dissuade consumers from using this composition.

The composition used according to the invention may be in any of the galenical forms normally used in the cosmetics field, and it may in particular be in the form of a suspension, dispersion, gel, serum, fluid or solid emulsion (W/O, O/W, multiple emulsion), aqueous, anhydrous or fatty stick, solid, liquid or pasty anhydrous product, loose or compacted powder, or cast, moulded or extruded form.

According to one particular mode, the composition is an emulsion.

According to one particular mode, the composition is a gel.

According to one particular mode, the composition is a solid anhydrous product.

According to one particular mode, the composition is a liquid or pasty anhydrous product.

According to one particular mode, the composition is a powder in loose or pressed form.

According to one particular mode, the composition is a cast product.

The term “anhydrous” is intended to mean that the composition used according to the invention has less than 5% by weight, in particular less than 3% by weight, more particularly less than 1% by weight of water or is even free of water.

The physiologically acceptable medium may also in particular comprise organic solvents, optionally water and/or oils, in particular for the compositions in fluid or pasty form.

According to another variant of the invention, the composition of the invention comprises an aqueous phase.

The aqueous phase comprises at least water and may also comprise organic solvents that are water-miscible (at ambient temperature: 25° C.), for instance monoalcohols containing from 2 to 6 carbon atoms, such as ethanol or isopropanol; polyols in particular containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, caprylyl glycol, dipropylene glycol or diethylene glycol; glycol ethers (in particular containing from 3 to 16 carbon atoms) such as mono-, di- or tripropylene glycol (C₁-C₄)alkyl ethers, mono-, di- or triethylene glycol (C₁-C₄)alkyl ethers, and mixtures thereof.

A water that is suitable for use in the invention may be a floral water such as cornflower water and/or a mineral water such as Vittel water, Lucas water or La Roche Posay water and/or a spring water.

The overall aqueous phase, while including all the hydrophilic substances of the composition that are capable of being dissolved in this same phase, is preferably present in the composition in a content preferably ranging from 5% to 98% by weight, more preferentially from 5% to 95% by weight, better still from 25% to 75% by weight, and more particularly from 25% to 60% by weight, relative to the total weight of said composition.

The aqueous phase may also comprise any water-soluble or water-dispersible compound that is compatible with an aqueous phase, such as gelling agents, film-forming polymers, thickeners or surfactants, and mixtures thereof.

According to another variant of the invention, the composition of the invention comprises a liquid fatty phase. Said phase is liquid (in the absence of structuring agent) at ambient temperature (20-25° C.). Preferentially, the water-immiscible organic liquid phase in accordance with the invention generally comprises at least one volatile oil and/or one non-volatile oil.

The term “oil” is intended to mean a fatty substance that is liquid at ambient temperature (25° C.) and atmospheric pressure (760 mmHg, i.e. 10⁵ Pa). The oil may be volatile or non-volatile.

For the purposes of the invention, the term “volatile oil” is intended to mean an oil that is capable of evaporating on contact with the skin or the keratin fibre in less than one hour, at ambient temperature and atmospheric pressure. The volatile oils of the invention are volatile cosmetic oils that are liquid at ambient temperature with a non-zero vapour pressure, at ambient temperature and atmospheric pressure ranging in particular from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “non-volatile oil” is intended to mean an oil that remains on the skin or the keratin fibre at ambient temperature and atmospheric pressure for at least several hours, and that in particular has a vapour pressure of less than 10⁻³ mmHg (0.13 Pa).

The oil may be chosen from any oil which is preferably physiologically acceptable, in particular mineral, animal, plant or synthetic oils; in particular volatile or non-volatile hydrocarbon-based oils and/or silicone oils and/or fluorinated oils, and mixtures thereof.

More precisely, the term “hydrocarbon-based oil” is intended to mean an oil mainly comprising carbon and hydrogen atoms and optionally one or more functions chosen from hydroxyl, ester, ether and carboxylic functions. Generally, the oil has a viscosity of from 0.5 to 100 000 mPa·s, preferably from 50 to 50 000 mPa·s and more preferably from 100 to 300 000 mPa·s.

Mention may be made, as examples of volatile oil which can be used in the invention, of:
volatile hydrocarbon-based oils chosen from hydrocarbon-based oils containing from 8 to 16 carbon atoms, and in particular C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C₈-C₁₆ esters and isohexyl neopentanoate, and mixtures thereof. Use may also be made of other volatile hydrocarbon-based oils, such as petroleum distillates, in particular those sold under the name Shell Solt by the company Shell; and volatile linear alkanes, such as those described in Patent Application DE10 2008 012 457 by the company Cognis.
volatile silicones, for instance linear or cyclic volatile silicone oils, in particular those with a viscosity of ≤8 centistokes (8×10⁻⁶ m²/s), and containing in particular from 2 to 7 silicon atoms, these silicones optionally comprising alkyl or alkoxy groups containing from 1 to 10 carbon atoms. As volatile silicone oils that may be used in the invention, mention may be made in particular of caprylyl methicone, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane;
and mixtures thereof.

Mention may be made, as examples of nonvolatile oil which can be used in the invention, of:
hydrocarbon-based oils of animal origin, such as perhydrosqualene;
hydrocarbon-based oils of plant origin, such as liquid triglycerides of fatty acids containing from 4 to 24 carbon atoms, for instance caprylic/capric acid triglycerides such as those sold by the company Stéarinerie Dubois or those sold under the names Miglyol 810, 812 and 818 by the company Dynamit Nobel;
linear or branched hydrocarbons, of mineral or synthetic origin, such as liquid paraffins and derivatives thereof, petroleum jelly, polydecenes, polybutenes, hydrogenated polyisobutene such as Parleam, or squalane;
synthetic ethers containing from 10 to 40 carbon atoms, such as dicaprylyl ether;
synthetic esters, in particular of fatty acids, isononyl isononanoate, isopropyl myristate, isopropyl palmitate, C₁₂-C₁₅ alcohol benzoates, hexyl laurate, diisopropyl adipate, 2-ethylhexyl palmitate, 2-octyldodecyl stearate, 2-octyldodecyl erucate, isostearyl isostearate;
fatty alcohols that are liquid at ambient temperature, containing a branched and/or unsaturated carbon-based chain containing from 12 to 26 carbon atoms, for instance octyldodecanol, isostearyl alcohol, 2-butyloctanol, 2-hexyldecanol, 2-undecylpentadecanol or oleyl alcohol;
higher fatty acids such as oleic acid, linoleic acid or linolenic acid;
carbonates such as dicapryl carbonate;
acetates;
citrates;
fluorinated oils that are optionally partially hydrocarbon-based and/or silicone-based, for instance fluorosilicone oils, fluoropolyethers and fluorosilicones as described in the document EP-A-847 752;
silicone oils, for instance non-volatile polydimethylsiloxanes (PDMSs); phenyl silicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates, and
mixtures thereof.

The oily phase may also comprise any liposoluble or lipodispersible compound that is compatible with an oily phase, such as gelling agents, film-forming polymers, thickeners, waxes or pasty substances, and mixtures thereof.

According to one particular form of the invention, the overall oily phase, including all the lipophilic substances of the composition capable of being dissolved in this same phase, represents from 5% to 95% by weight and preferentially from 10% to 80% by weight, relative to the total weight of the composition.

### Emulsions

According to one particular mode, the compositions according to the invention may be in the form of an emulsion such as an oil-in-water emulsion (continuous aqueous phase in which is dispersed an oily phase in the form of droplets so as to obtain a macroscopically homogeneous mixture) or a water-in-oil emulsion (continuous oily phase in which is dispersed an aqueous phase in the form of droplets so as to obtain a macroscopically homogeneous mixture).

In the case where the composition is in emulsion form, it generally comprises one or more emulsifying, preferably nonionic, surfactants.

The emulsifiers are chosen in an appropriate manner according to the emulsion to be obtained (W/O or O/W emulsion).

Examples of emulsifiers that may be mentioned for the O/W emulsions include nonionic surfactants, and in particular esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives comprising oxyethylene and/or oxypropylene units, such as glyceryl esters of C₈-C₂₄ fatty acids, and the oxyalkylenated derivatives thereof; polyethylene glycol esters of C₈-C₂₄ fatty acids, and the oxyalkylenated derivatives thereof; sorbitol esters of C₈-C₂₄ fatty acids, and the oxyalkylenated derivatives thereof; and the oxyalkylenated derivatives thereof; fatty alcohol ethers; sugar esters of C₈-C₂₄ fatty acids, sugar ethers of C₈-C₂₄ fatty alcohols, and mixtures thereof.

Mention may in particular be made, as glyceryl ester of fatty acid, of glyceryl stearate (glyceryl monostearate, distearate and/or tristearate) (CTFA name: glyceryl stearate) or glyceryl ricinoleate, and mixtures thereof.

Polyethylene glycol esters of fatty acids that may in particular be mentioned include polyethylene glycol stearate (polyethylene glycol monostearate, distearate and/or tristearate) and more particularly polyethylene glycol 40 OE monostearate (CTFA name: PEG-40 stearate) and polyethylene glycol 50 OE monostearate (CTFA name: PEG-50 stearate) and polyethylene glycol 100 OE monostearate (CTFA name: PEG-100 stearate) and mixtures thereof.

Mixtures of these surfactants may also be used, for instance the product containing glyceryl stearate and PEG-100 stearate, sold under the name Arlacel 165® by the company Uniqema, and the product containing glyceryl stearate (glyceryl mono-distearate) and potassium stearate, sold under the name Tegin® by the company Goldschmidt (CTFA name: glyceryl stearate SE).

Examples of fatty alcohol ethers that may be mentioned include polyethylene glycol ethers of fatty alcohols containing from 8 to 30 carbon atoms and in particular from 10 to 22 carbon atoms, such as polyethylene glycol ethers of cetyl alcohol, of stearyl alcohol or of cetearyl alcohol (mixture of cetyl alcohol and stearyl alcohol). Mention may be made, for example, of ethers containing from 1 to 200 and preferably from 2 to 100 oxyethylene groups, such as those with the CTFA name Ceteareth-20 or Ceteareth-30, and mixtures thereof.

Examples of sugar mono- or polyalkyl esters or ethers that may be mentioned include the methylglucose isostearate sold under the name Isolan-IS® by the company Degussa Goldschmidt, or else the sucrose distearate sold under the name Crodesta F50® by the company Croda, and the sucrose stearate sold under the name Ryoto sugar ester S 1570® by the company Mitsubishi Kagaku Foods; sugar esters such as sucrose stearate; sugar ethers of fatty alcohols, in particular alkylpolyglucosides (APGs) such as decyl glucoside and lauryl glucoside sold, for example, by the company Henkel under the respective names Plantaren 2000® and Plantaren 1200®, cetostearyl glucoside as a mixture with cetostearyl alcohol, sold, for example, under the name Montanov 68® by the company SEPPIC, under the name Tegocare CG90® by the company Goldschmidt and under the name Emulgade KE3302® by the company Henkel, and also arachidyl glucoside, for example in the form of the mixture of arachidyl and behenyl alcohols and of arachidyl glucoside, sold under the name Montanov 202® by the company SEPPIC.

Examples of W/O emulsifying surfactants that may be mentioned include alkyl esters or ethers of sorbitan, of glycerol, of polyol or of sugars; silicone surfactants, for instance dimethicone copolyols, such as the one having the INCI name Dimethicone (and) PEG/PPG-18/18 Dimethicone sold under the brand x-22-6711D® by the company Shin Etsu, the mixture of cyclomethicone and of dimethicone copolyol, sold under the name DC 5225 C® by the company Dow Corning, and alkyldimethicone copolyols such as the laurylmethicone copolyol sold under the name Dow Corning 5200 Formulation Aid by the company Dow Corning; cetyl dimethicone copolyol, for instance cetyl PEG/PPG-10/1 dimethicone, such as the product sold under the name Abil EM 90® by the company Evonik Goldschmidt, and the mixture of cetyl dimethicone copolyol, of polyglyceryl isostearate (4 mol) and of hexyl laurate, sold under the name Abil WE 09® by the company Goldschmidt. One or more coemulsifiers, which may be chosen advantageously from the group comprising polyol alkyl esters, may also be added thereto.

Mention may also be made of non-silicone emulsifying surfactants, in particular alkyl esters or ethers of sorbitan, of glycerol, of polyol or of sugars.

Polyol alkyl esters that may in particular be mentioned include polyethylene glycol esters, for instance PEG-30 dipolyhydroxystearate, such as the product sold under the name Arlacel P135® by the company ICI.

Examples of glycerol and/or sorbitan esters that may be mentioned include polyglyceryl isostearate (i.e.: polyglyceryl-4 isostearate), such as the product sold under the name Isolan GI 34® by the company Evonik Goldschmidt; sorbitan isostearate, such as the product sold under the name Arlacel 987® by the company ICI; sorbitan glyceryl isostearate, such as the product sold under the name Arlacel 986® by the company ICI, and mixtures thereof.

Mention may also be made of emulsifying polyoxyalkylenated silicone elastomers, such as those mentioned in U.S. Pat. No. 5,236,986, U.S. Pat. No. 5,412,004, U.S. Pat. No. 5,837,793 and U.S. Pat. No. 5,811,487. These silicone elastomers are preferably formulated in the form of a gel in a hydrocarbon-based oil and/or a silicone oil. In these gels, the polyoxyalkylenated silicone elastomer is often in the form of spherical particles.

By way of example of polyoxyalkenylated silicone elastomers, mention may be made of those sold by the company Shin Etsu with the following names:
KSG-21® (containing 27% active material) having the INCI name: Dimethicone/PEG-10 Dimethicone Vinyl Dimethicone crosspolymer,
KSG-20® (containing 95% active material) having the INCI name: PEG-10 Dimethicone Crosspolymer,
KSG-30® (containing 100% active material) having the INCI name: Lauryl PEG-Dimethicone Vinyl Dimethicone Crosspolymer,
KSG-31® (containing 25% active material) having the INCI name: Lauryl PEG-15 Dimethicone Vinyl Dimethicone Crosspolymer,
KSG-32® or KSG-42 or KSG-320 ou KSG-30 (containing 25% active material) having the INCI name: Lauryl PEG-15 Dimethicone Vinyl Dimethicone Crosspolymer,
KSG-33 (containing 20% active material),
KSG-210® (containing 25% active material) having the INCI name: Dimethicone/PEG-10/15 Crosspolymer,
KSG-310®: polyoxyethylenated lauryl polydimethylsiloxane in a mineral oil,
KSG-330®,
KSG-340®,
X-226146® (containing 32% active material) having the INCI name: Dimethicone/PEG-10 Dimethicone Vinyl Dimethicone crosspolymer.

Mention may also be made of those sold by the company Dow Corning under the trade names:
DC9010® (containing 9% active material) having the INCI name: PEG-12 Dimethicone Crosspolymer,
DC9011® containing 11% active material.

Mention may also be made, among water/oil emulsifiers, of polyglycerolated silicone elastomers, such as those described in document WO-A-2004/024798. As examples of silicone elastomers, mention may be made of those sold by the company Shin Etsu, with the following trade names:
KSG-710® (containing 25% active material) having the INCI name: Dimethicone/Polyglycerin-3 Crosspolymer,
KSG-810®,
KSG-820®,
KSG-830®,
KSG-840®.

Use will more preferentially be made of a W/O emulsifier chosen from polyglyceryl-4 isostearate, cetyl PEG/PPG-10/1 dimethicone, dimethicone (and) PEG/PPG-18/18 dimethicone, dimethicone/PEG-10/15 crosspolymer, dimethicone/polyglycerin-3 crosspolymer and mixtures thereof.

According to one particular mode, the composition of the invention is a water-in-oil emulsion.

The compositions according to the invention may also contain additional cosmetic ingredients conventionally used for the formulation of particular galenics, generally adjusted with regard to the intended keratin material. This or these additional cosmetic ingredient(s) may in particular be chosen from waxes, pasty fatty substances, film-forming polymers, nonionic, anionic or cationic surfactants, hydrophilic or lipophilic gelling agents, dispersants, hydrophilic or lipophilic active agents, preservatives, antioxidants, solvents, fragrances, fillers other than the soft-focus fillers previously defined, sunscreens, bactericides, odour absorbers, additional colorants other than the composite pigments previously defined (for example: pigments, nacres, water-soluble or liposoluble dyes), salts, and mixtures thereof.

Thus, a cosmetic composition of the invention may comprise, in addition to the soft-focus filler(s) and the composite pigment(s) defined above, at least one additional cosmetic ingredient chosen from waxes, pasty fatty substances, film-forming polymers, nonionic, anionic or cationic surfactants, hydrophilic or lipophilic gelling agents, dispersants, hydrophilic or lipophilic active agents, preservatives, antioxidants, solvents, fragrances, fillers other than the soft-focus fillers previously defined, sunscreens, bactericides, odour absorbers, additional colorants other than the composite pigments previously defined (for example: pigments, nacres, water-soluble or liposoluble dyes), salts, and mixtures thereof.

The amounts of the additional cosmetic ingredients are those conventionally used in the field under consideration, for example from 0.01% to 20% of the total weight of the composition and preferably from 0.01% to 10% of the total weight of the composition.

Needless to say, those skilled in the art will take care to select the optional additional ingredients and/or the amount thereof such that the advantageous properties of the composition according to the invention are not, or are not substantially, adversely affected by the envisaged addition.

### Dispersant

Advantageously, a composition according to the invention may also comprise a dispersant.

Such a dispersant may be a surfactant, an oligomer, a polymer or a mixture of several thereof.

According to one particular embodiment, a dispersant in accordance with the invention is a surfactant.

According to a particular embodiment variant, a composition according to the invention comprises less than 1% by weight of surfactant relative to the total weight of the composition, or even is free of surfactant.

### Active Agent

For a care application in particular, a composition according to the invention may comprise at least one moisturizer (also known as a humectant).

Preferably, the moisturizer is glycerol.

The moisturizer(s) may be present in the composition in a content ranging from 0.1% to 15% by weight, in particular from 0.5% to 10% by weight or even from 1% to 6% by weight relative to the total weight of said composition.

As other active agents that may be used in the composition of the invention, examples that may be mentioned include vitamins and sunscreens, and mixtures thereof.

Preferably, a composition according to the invention comprises at least one active agent.

It is a matter of routine operations for those skilled in the art to adjust the nature and the amount of the additives present in the compositions in accordance with the invention such that the desired cosmetic properties thereof are not thereby affected.

According to one embodiment, a composition of the invention may advantageously be in the form of a composition for caring for the skin of the body or the face, in particular the face, for making the imperfections uniform with a natural result.

According to another embodiment, a composition of the invention may advantageously be in the form of a makeup base composition.

According to another embodiment, a composition of the invention may advantageously be in the form of a foundation.

According to another embodiment, a composition of the invention may advantageously be in the form of a nail makeup and/or care product for hiding and/or smoothing out relief and colour imperfections.

According to one embodiment, a composition of the invention may advantageously be in the form of a makeup product for the area around the eyes, the shadows under the eyes or the eyelids, such as an eyeshadow, or a concealer product. It may be in the form of a loose or pressed powder, cream or gel.

According to one embodiment, a composition of the invention may advantageously be in the form of a lip makeup and/or care composition for hiding and/or smoothing out relief and colour imperfections, in particular in the form of sticks or a gloss.

Such compositions are in particular prepared according to the general knowledge of those skilled in the art.

The expressions “between . . . and . . . ” and “ranging from . . . to . . . ” should be understood as being inclusive of the limits, unless otherwise specified.

The invention is illustrated in greater detail by the examples and figures presented below. Unless otherwise mentioned, the amounts indicated are expressed as weight percentages relative to the total weight of the composition.

### EXAMPLES

### Examples 1 and 2

Two foundations in water-in-oil emulsion form 1 and 2 were prepared. The first contained titanium dioxide, iron oxide and the soft-focus filler and the second contained the composite pigments of the invention and the soft-focus filler.

The contrast ratio was measured according to the method indicated above of Examples 1 and 2. In order to be able to compare the accentuation of the wrinkles and fine lines of the two formulations 1 and 2, the hybrid pigment concentration of composition 2 was chosen so as to obtain a contrast ratio (CR) equivalent to that of composition 1 (% CR±5).

### White Base:

| | | White |
|---|---|---|
| Phase | Ingredients | Base |
| A1 | CETYL PEG/PPG-10/1 DIMETHICONE | 3.88 |
| | (ABIL EM90 ® from EVONIK GOLDSCHMIDT) | |
| | POLYGLYCEROLATED ISOSTEARATE (4 mol) | 1.30 |
| | (ISOLAN GI-34) | |
| | 2-ETHYLHEXYL 4-METHOXYCINNAMATE | 9.74 |
| | (PARSOL MCX ®) | |
| | PHENYL TRIMETHICONE | 9.74 |
| | DIMETHICONE (and) PEG/PPG-18/18 | 1.40 |
| | DIMETHICONE (X-22-6711D ® from | |
| | SHIN ETSU) | |
| | DIMETHICONE (KF-96L-2CS from SHIN ETSU) | 6.68 |
| | CYCLOHEXASILOXANE | 5.34 |
| | DIMETHICONE (XIAMETER PMX-200 SILICONE | 12.03 |
| | FLUID 5CS from DOW CORNING) | |
| A2 | DIMETHICONE (and) DIMETHICONE/PEG-10/15 | 0.69 |
| | CROSSPOLYMER | |
| | DIMETHICONE (and) | 0.59 |
| | DIMETHICONE/POLYGLYCERIN-3 | |
| | CROSSPOLYMER | |
| B1 | DEMINERALIZED WATER | qs 100 |
| | BUTYLENE GLYCOL | 6.48 |
| | MAGNESIUM SULFATE | 0.89 |
| | PROPANEDIOL | 6.48 |
| | ADENOSINE | 0.06 |
| | SORBITOL | 0.65 |
| | PHENOXYETHANOL | 0.90 |
| D1 | DENAT. ALCOHOL | 6.48 |

| | | Example 1 | |
|---|---|---|---|
| | | (outside the | Example 2 |
| Phase | Ingredients | invention) | (invention) |
| White base | | qs 100 | qs 100 |
| C1 | ALUMINA (and) TITANIUM OXIDE | — | 13.20 |
| | (and) TRIETHOXYCAPRYLYLSILANE | | |
| | (COVALUMINE ATLAS WHITE AS ® | | |
| | from SENSIENT) | | |
| | ALUMINA (and) CI 77491 (and) | — | 0.73 |
| | TRIETHOXYCAPRYLYLSILANE | | |
| | (COVALUMINE SONOMA RED AS ® | | |
| | from SENSIENT) | | |
| | ALUMINA (and) CI 77499 (and) | — | 0.23 |
| | TRIETHOXYCAPRYLYLSILANE (and) | | |
| | SILICA | | |
| | (COVALUMINE SONOMA BLACK AS ® | | |
| | from SENSIENT) | | |
| | ALUMINA (and) CI 77492 (and) | — | 3 |
| | TRIETHOXYCAPRYLYLSILANE | | |
| | (COVALUMINE SONOMA YELLOW AS ® | | |
| | from SENSIENT) | | |
| | ANATASE TITANIUM OXIDE COATED | 8.63 | — |
| | WITH ALUMINIUM STEAROYL | | |
| | GLUTAMATE (97/3) (CI: 77891) | | |
| | (NAI-TAO-77891 ® from Myoshi Kasei) | | |
| | YELLOW IRON OXIDE COATED WITH | 1.91 | — |
| | ALUMINIUM STEAROYL GLUTAMATE | | |
| | (3%) | | |
| | (NAI-C33-8001-10 ® from Myoshi | | |
| | Kasei) | | |
| | BLACK IRON OXIDE COATED WITH | 0.20 | — |
| | ALUMINIUM STEAROYL GLUTAMATE | | |
| | (3%) | | |
| | (NAI-C33-7001-10 ® from Myoshi | | |
| | Kasei) | | |
| | RED IRON OXIDE COATED WITH | 0.48 | — |
| | ALUMINIUM STEAROYL GLUTAMATE | | |
| | (3%) | | |
| | (NAI-C33-9001-10 ® from Myoshi | | |
| | Kasei) | | |
| E1 | ACRYLATES/ETHYLHEXYL | 12.00 | 12.00 |
| | ACRYLATE CROSSPOLYMER | | |
| | (MAKIBEADS SP10 ® from DAITO | | |
| | KASEI) | | |

| | | |
|---|---|---|
| Contrast Ratio | 72.6% | 71.10% |

### Preparation of the White Base:

Phase A1 was weighed out in a main beaker, which was placed on a Moritz stirrer at ambient temperature (20-25° C.). Phase A2 was weighed out and was added to phase A1 with stirring at 4500 rpm for 10 minutes. The demineralized water was made to boil and phase B1 was weighed out in a beaker. It was placed on a magnetic plate with a magnetic bar. Phase B1 was poured into phase A(A1+A2) with strong stirring at 4500 rpm for 10 minutes. Phase D1 was added and the mixture was left to stir for 2 minutes.

Compositions 1 to 2 were Prepared According to the Following Protocol:

Phase C1 was weighed out and added to the white base with stirring on a Moritz stirrer. Phase E1 was added with mild stirring.

### Correction of the Accentuation

Examples 1 and 2 were evaluated by expert beauticians. The evaluation relates to 6 women. After makeup removal, the beautician applies 0.10 ml to half of the face and evaluates the makeup result after 4 minutes of drying.

The two formulae 1 and 2 have the same colour imperfection correction performance.

For this same level of correction, the formula describing the invention accentuates the wrinkles and fine lines on the face much less.

### Natural Makeup Result:

An evaluation of Example 2 according to the invention, of the natural appearance after application of Examples 1 and 2, was carried out by consumers. The test was carried out on 2 targets:

8 women from 18 to 35 years old, in search of a compromise between coverage of the imperfections and a natural result.

9 women who gave up complexion products because of the non-natural result. The women used the composition at home for 6 days. Individual interviews regarding the foundation were then carried out in order to obtain their evaluation. The results showed that formulation 2 according to the invention provides sufficient coverage while at the same time giving a natural makeup result. The women judged that the foundation attenuated their colour and relief imperfections without a sensation of thickness usually felt with the prior art foundations.

### Example 3: W/O Emulsion Foundations

Example 3 was prepared under the same operating conditions as Examples 1 and 2.

| | Example 3 |
|---|---|
| Ingredients | (invention) |
| MAGNESIUM SULFATE | 0.6 |
| ADENOSINE | 0.04 |
| SORBITOL AS AN AQUEOUS 70% SOLUTION | 0.5 |
| ALUMINA (and) TITANIUM OXIDE (and) | 14.1 |
| TRIETHOXYCAPRYLYLSILANE | |
| (COVALUMINE ATLAS WHITE AS® from | |
| SENSIENT) | |
| ALUMINA (and) CI 77491 (and) | 0.75 |
| TRIETHOXYCAPRYLYLSILANE | |
| (COVALUMINE SONOMA RED AS ® from | |
| SENSIENT) | |
| ALUMINA (and) CI 77492 (and) | 2.20 |
| TRIETHOXYCAPRYLYLSILANE | |
| (COVALUMINE SONOMA YELLOW AS ® from | |
| SENSIENT) | |
| ALUMINA (and) CI 77499 (and) | 0.11 |
| TRIETHOXYCAPRYLYLSILANE (and) SILICA | |
| (COVALUMINE SONOMA BLACK AS ® from | |
| SENSIENT) | |
| 2-PHENOXYETHANOL | 0.70 |
| 2-ETHYLHEXYL 4-METHOXYCINNAMATE | 7.00 |
| (PARSOL MCX ®) | |
| ACRYLATES/ETHYLHEXYL ACRYLATE | 12.00 |
| CROSSPOLYMER (MAKIBEADS SP10 ® from | |
| DAITO KASEI) | |
| PHENYL TRIMETHICONE | 7.0 |
| DODECAMETHYLPENTASILOXANE | 4.70 |
| CYCLOHEXASILOXANE | 3.70 |
| DIMETHICONE | 9.00 |
| CETYL PEG/PPG-10/1 DIMETHICONE | 2.80 |
| (ABIL EM90 ® from EVONIK GOLDSCHMIDT) | |
| DIMETHICONE (and) DIMETHICONE/PEG- | 0.50 |
| 10/15 CROSSPOLYMER | |
| (KSG-210 ® from Shin Etsu) | |
| DIMETHICONE (and) | 0.40 |
| DIMETHICONE/POLYGLYCERIN-3 | |
| CROSSPOLYMER | |
| (KSG 710 ® from Shin Etsu) | |
| DIMETHICONE (and) PEG/PPG-18/18 | 1.00 |
| DIMETHICONE | |
| (X-22-6711D ® from SHIN ETSU) | |
| POLYGLYCERYL-4 ISOSTEARATE | 0.90 |
| BUTYLENE GLYCOL | 4.60 |
| PROPANEDIOL | 4.60 |
| DENATURED ALCOHOL | 4.60 |
| DEMINERALIZED WATER | qs 100 |

These formulae were evaluated by an expert beautician on a panel of 6 women. The beautician applies 0.10 ml to half of the face and evaluates the makeup result after 4 minutes of drying. The following criteria were evaluated on a scale of 0 to 36:

Accentuation of wrinkles/fine lines
Smoothing out of wrinkles/fine lines
Accentuation of the skin grain
Smoothing out of the skin grain

The results obtained are indicated in the following table:

| | Accentuation | Smoothing | | |
|---|---|---|---|---|
| | of | out of | Accentuation | Smoothing |
| | wrinkles/fine | wrinkles/fine | of the skin | out of the |
| Composition | lines | lines | grain | skin grain |
| EXAMPLE 3 | 0 | 8 | 0 | 22 |
| (invention) | | | | |

The results showed that formula 3 of the invention does not exhibit any accentuation and gives a high level of smoothing out of the skin grain.

### Example 4: Loose Powder

| Ingredients | Amounts in % by weight |
|---|---|
| TALC (LUZENAC 00 from IMERYS) | qs 100 |
| METHYL METHACRYLATE | 24.00 |
| CROSSPOLYMER | |
| (COVABEAD LH 85 from Sensient) | |
| ALUMINA (and) TITANIUM OXIDE (and) | 28.08 |
| TRIETHOXYCAPRYLYLSILANE | |
| (COVALUMINE ATLAS WHITE AS ® from | |
| SENSIENT) | |
| ALUMINA (and) IRON OXIDES (and) | 1.50 |
| TRIETHOXYCAPRYLYLSILANE | |
| (COVALUMINE SONOMA RED AS from | |
| Sensient) | |
| ALUMINA (and) IRON OXIDES (and) | 2.20 |
| TRIETHOXYCAPRYLYLSILANE | |
| (COVALUMINE SONOMA YELLOW AS | |
| from Sensient) | |
| ALUMINA (and) IRON OXIDES (and) | 0.22 |
| TRIETHOXYCAPRYLYLSILANE | |
| (COVALUMINE SONOMA BLACK AS from | |
| Sensient) | |

The composition makes it possible to effectively conceal the skin imperfections, without any relief accentuation effect or mask effect, and to obtain good uniformity of the complexion and a natural appearance.

### Example 5: Contour of the Eye: Water-in-Oil Emulsion

| | Amounts |
|---|---|
| | in % by |
| Ingredients | weight |
| MAGNESIUM SULFATE | 0.6 |
| CHLORPHENESIN | 0.2 |
| PHENOXYETHANOL | 0.5 |
| CETYL PEG/PPG-10/1 DIMETHICONE | 1.7 |
| ACETYLATED GLYCOL STEARATE | 0.5 |
| HEXYL LAURATE | 1.7 |
| CYCLOPENTASILOXANE | 13 |
| 2-OLEAMIDO-1,3-OCTADECANEDIOL | 0.03 |
| PROPYLENE GLYCOL | 4.5 |
| DIMETHICONE | 2.6 |
| DENAT. ALCOHOL | 0.2 |
| WATER AQUA | qs 100 |
| POLYGLYCERYL-4 ISOSTEARATE | 2.3 |
| ISODODECANE | 1.7 |
| ALUMINA (AND) IRON OXIDES (AND) | 0.2 |
| TRIETHOXYCAPRYLYLSILANE | |
| (COVALUMINE SONOMA RED AS ® FROM SENSIENT) | |
| ALUMINA (AND) IRON OXIDES (AND) | 0.6 |
| TRIETHOXYCAPRYLYLSILANE | |
| (COVALUMINE SONOMA YELLOW AS ® FROM | |
| SENSIENT) | |
| ALUMINA (AND) IRON OXIDES (AND) | 0.03 |
| TRIETHOXYCAPRYLYLSILANE | |
| (COVALUMINE SONOMA BLACK AS ® - SENSIENT) | |
| ALUMINA (AND) TITANIUM DIOXIDE (AND) | 3 |
| TRIETHOXYCAPRYLYLSILANE (COVALUMINE | |
| ATLAS WHITE AS ® - SENSIENT) | |
| CROSSLINKED POLYDIMETHYLSILOXANE GUM | 12 |
| BEADS COATED WITH SILSESQUIOXANE RESIN | |
| (92/8) (KSP 100 ® - SHIN ETSU) | |
| NYLON-12 (ORGASOL 2002 EXD NAT COS FROM | 6 |
| ARKEMA) | |
| METHYLPARABEN | 0.2 |

The composition makes it possible to effectively conceal the imperfections of the skin around the eye, without any relief accentuation effect or mask effect, and to obtain good uniformity of the complexion and a natural appearance.

### Example 6: Lipstick

| | Amount |
|---|---|
| | in % by |
| Ingredients | weight |
| PRUNUS AMYGDALUS DULCIS (SWEET ALMOND) OIL | 7.33 |
| CAPRYLIC/CAPRIC TRIGLYCERIDE | qs 100 |
| HYDROGENATED COCO-GLYCERIDES | 2.68 |
| ISOPROPYL ISOSTEARATE | 7 |
| EUPHORBIA CERIFERA (CANDELILLA) WAX | 11 |
| CANOLA OIL | 6.5 |
| HYDROGENATED MYRISTYL OLIVE ESTERS | 1.94 |
| HYDROGENATED STEARYL OLIVE ESTERS | 1.94 |
| BUTYROSPERMUM PARKII (SHEA BUTTER) | 15 |
| ORYZA SATIVA (RICE) STARCH | 0.98 |
| SUCROSE ACETATE ISOBUTYRATE | 20 |
| ALUMINA (and) IRON OXIDES (and) | 4 |
| TRIETHOXYCAPRYLYLSILANE | |
| (COVALUMINE SONOMA RED AS ® - Sensient) | |
| ALUMINA (and) IRON OXIDES (and) | 0.5 |
| TRIETHOXYCAPRYLYLSILANE | |
| (COVALUMINE SONOMA YELLOW AS ® - Sensient) | |
| ALUMINA (and) TITANIUM DIOXIDE (and) | 3.8 |
| TRIETHOXYCAPRYLYLSILANE | |
| (COVALUMINE ATLAS WHITE AS ® - SENSIENT) | |
| ACRYLATES/ETHYLHEXYL ACRYLATE COPOLYMER - | 10 |
| 12-18 MICRON MICROSPHERES (TECHPOLYMER | |
| ACP-8C ® - SEKISUI PLASTICS) | |

The composition makes it possible to effectively conceal the imperfections, without any relief accentuation effect, and to obtain good uniformity of the product on the lips.

### Example 7: Eyeshadow

| Chemical name | % Concentration |
|---|---|
| SYNTHETIC SERICITE | 3.351 |
| (FLUOROPHLOGOPITE) | |
| TALC (LUZENAC 00 from IMERYS) | 25.8027 |
| MAGNESIUM STEARATE | 4.9953 |
| GLYCERYL TRIISOSTEARATE | 6 |
| PHENYLTRIMETHYLSILOXYTRISILOXANE | qs 100 |
| (VISCOSITY: 20 CST - MW: 372) | |
| 1,2-OCTANEDIOL | 0.5 |
| YELLOW IRON OXIDE (CI 77492) ON | 15 |
| ALUMINA, COATED WITH | |
| TRIETHOXYCAPRYLYLSILANE | |
| (COVALUMINE SONOMA YELLOW AS ® | |
| SENSIENT) | |
| TITANIUM DIOXIDE (CI 77891) ON | 3.351 |
| ALUMINA COATED WITH | |
| TRIETHOXYCAPRYLYLSILANE | |
| (COVALUMINE ATLAS WHITE AS ® - | |
| SENSIENT) | |
| MICRONIZED | 20 |
| TRIETHOXYCAPRYLYLSILANE COATED | |
| WITH RED IRON OXIDE (CI 77491) ON | |
| ALUMINA (COVALUMINE SONOMA RED | |
| AS ® - SENSIENT) | |
| HOLLOW SPHERES OF COPOLYMER OF | 15 |
| ACRYLATES/ETHYLHEXYL ACRYLATE | |
| CROSSPOLYMER (MAKIBEADS SP-10 ® - | |
| DAITO KASEI KOGYO) | |

The composition makes it possible to effectively conceal the imperfections, without any relief accentuation effect, and to obtain good uniformity of the product on the eyelids and a natural appearance.

### Example 8: Nail Varnish

| Chemical name | % Concentration |
|---|---|
| NITROCELLULOSE | 11 |
| N-ETHYL O,P-TOLUENESULFONAMIDE | 5 |
| ALKYD RESIN | 10 |
| ISOPROPANOL | 4 |
| BUTYL ACETATE/ETHYL ACETATE 50/50 | qs 100 |
| ALUMINA (AND) IRON OXIDES (AND) | 6 |
| TRIETHOXYCAPRYLYLSILANE | |
| (COVALUMINE SONOMA RED AS ® - | |
| SENSIENT) | |
| ALUMINA (AND) IRON OXIDES (AND) | 0.5 |
| TRIETHOXYCAPRYLYLSILANE | |
| (COVALUMINE SONOMA YELLOW AS ® | |
| SENSIENT) | |
| ALUMINA (AND) TITANIUM DIOXIDE (AND) | 2.8 |
| TRIETHOXYCAPRYLYLSILANE(COVALUMINE | |
| ATLAS WHITE AS ® (SENSIENT) | |
| ACRYLATES/ETHYLHEXYL ACRYLATE | 9 |
| COPOLYMER - 12-18 MICRON | |
| MICROSPHERES (TECHPOLYMER ACP-8C ®) | |
| (SEKISUI PLASTICS) | |

The composition makes it possible to effectively conceal the imperfections, without any relief accentuation effect, and to obtain good uniformity of the product on the nails.

### Examples 9 to 11: Water-in-Oil Emulsions

Formulation 9 according to the invention was prepared, as were two other comparative compositions (Examples 10 and 11) with conventional pigments and a soft-focus filler and not containing boron nitride. The concentration of conventional pigments was chosen so as to obtain a contrast ratio equivalent to that of composition 1 (% CR±5) in order to be able to compare their smoothing out and non-accentuation performance.

### White Base:

| Phase | Ingredients INCI name | White Base |
|---|---|---|
| A1 | CETYL PEG/PPG-10/1 DIMETHICONE | 5.51 |
| | (ABIL EM90 ® from EVONIK GOLDSCHMIDT) | |
| | POLYGLYCERYL-4 ISOSTEARATE | 1.84 |
| | ETHYLHEXYL METHOXYCINNAMATE | 9.54 |
| | (PARSOL MCX ® from DSM) | |
| | PHENYL TRIMETHICONE | 13.81 |
| | DIMETHICONE (and) PEG/PPG-18/18 | 1.99 |
| | DIMETHICONE | |
| | DIMETHICONE | 9.47 |
| | (XIAMETER PMX-200 SILICONE FLUID 2CS) | |
| | CYCLOHEXASILOXANE | 7.57 |
| | DIMETHICONE | 17.05 |
| | (DOW CORNING TORAY SH200 C FLUID 5CS) | |
| B1 | WATER | qs 100 |
| | BUTYLENE GLYCOL | 3.82 |
| | MAGNESIUM SULFATE | 0.96 |
| | PROPANEDIOL | 3.82 |
| | ADENOSINE | 0.05 |
| | SORBITOL | 0.70 |
| | PHENOXYETHANOL | 0.95 |

| | | Example 9 | Comparative | Comparative |
|---|---|---|---|---|
| Phase | Ingredients INCI name | (invention) | Example 10 | Example 11 |
| White Base | | qs 100 | qs 100 | qs 100 |
| C1 | ALUMINA (and) TITANIUM | 14.12 | — | — |
| | DIOXIDE (and) | | | |
| | TRIETHOXYCAPRYLYLSILANE | | | |
| | (COVALUMINE ATLAS WHITE | | | |
| | AS ® from SENSIENT) | | | |
| | ALUMINA (and) IRON OXIDES | 0.75 | — | — |
| | (and) | | | |
| | TRIETHOXYCAPRYLYLSILANE | | | |
| | (COVALUMINE SONOMA RED | | | |
| | AS ® from SENSIENT) | | | |
| | ALUMINA (and) IRON OXIDES | 0.11 | — | — |
| | (and) | | | |
| | TRIETHOXYCAPRYLYLSILANE | | | |
| | (COVALUMINE SONOMA BLACK | | | |
| | AS ® from SENSIENT) | | | |
| | ALUMINA (and) IRON OXIDES | 2.18 | — | — |
| | (and) | | | |
| | TRIETHOXYCAPRYLYLSILANE | | | |
| | (COVALUMINE SONOMA | | | |
| | YELLOW AS ® from SENSIENT) | | | |
| | TITANIUM DIOXIDE (and) | — | — | 6.98 |
| | DISODIUM STEAROYL | | | |
| | GLUTAMATE (and) ALUMINIUM | | | |
| | HYDROXIDE | | | |
| | NAI-TAO-77891 from MIYOSHI | | | |
| | KASEI | | | |
| | IRON OXIDES (and) DISODIUM | — | | 0.32 |
| | STEAROYL GLUTAMATE (and) | | | |
| | ALUMINIUM HYDROXIDE | | | |
| | NAI-C33-8001-10 from MIYOSHI | | | |
| | KASEI | | | |
| | IRON OXIDES (and) DISODIUM | — | | 0.13 |
| | STEAROYL GLUTAMATE (and) | | | |
| | ALUMINIUM HYDROXIDE | | | |
| | NAI-C33-7001-10 from MIYOSHI | | | |
| | KASEI | | | |
| | IRON OXIDES (and) DISODIUM | — | | 1.58 |
| | STEAROYL GLUTAMATE (and) | | | |
| | ALUMINIUM HYDROXIDE | | | |
| | NAI-C33-9001-10 from MIYOSHI | | | |
| | KASEI | | | |
| | SYNTHETIC | — | — | 1.00 |
| | FLUORPHLOGOPITE | | | |
| | TITANIUM DIOXIDE (and) | — | 8.15 | — |
| | TRIETHOXYCAPRYLYLSILANE | | | |
| | UNIPURE WHITE LC 981 AS-EM | | | |
| | from Sensient | | | |
| | IRON OXIDES (and) IRON | — | 0.41 | — |
| | OXIDES (and) | | | |
| | TRIETHOXYCAPRYLYLSILANE | | | |
| | UNIPURE RED LC381 AS EM | | | |
| | from Sensient | | | |
| | IRON OXIDES (and) | — | 0.15 | — |
| | TRIETHOXYCAPRYLYLSILANE | | | |
| | UNIPURE BLACK LC989 AS-EM | | | |
| | IRON OXIDES (and) | — | 1.31 | — |
| | TRIETHOXYCAPRYLYLSILANE | | | |
| | UNIPURE YELLOW LC182 AS | | | |
| | EM from Sensient | | | |
| D1 | DENATURED ALCOHOL | 4.60 | 4.60 | 4.60 |
| E1 | METHYL METHACRYLATE | 8.00 | 12.00 | 12.00 |
| | CROSSPOLYMER (COVABEAD | | | |
| | LH 85 from Sensient) | | | |
| | BORON NITRIDE (PUHP 1030L | 4.00 | — | — |
| | from SAINT GOBAIN CERAMICS) | | | |

| | | | |
|---|---|---|---|
| Contrast Ratio % CR | 72.8 | 69.5 | 70.4 |

### Preparation Protocol

a) Protocol for Preparing the White Base:

A1 was weighed out in the main beaker, placed on a Moritz stirrer at ambient temperature. The demineralized water was made to boil and B1 was weighed out in a beaker. It was placed on a magnetic plate with a magnetic bar. B1 was poured into A1 with strong stirring at 4500 rpm for 10 minutes.

b) Protocol for Preparing the Formulae of Examples 9, 10 and 11:

E1 was added with mild stirring. C1 was weighed out and added to (White Base+E1) with stirring on a Moritz stirrer at ambient temperature at 4500 rpm for 10 min. D1 was added and the mixture was left to stir for 2 minutes.

These formulae were evaluated by an expert beautician on a panel of 6 women. The beautician applies 0.10 ml to half of the face and evaluates the makeup result after 4 minutes of drying.

The following criteria were evaluated on a scale of 0 to 36

Effects on wrinkles/fine lines
Smoothing effects Dilated pores
“Colour” corrector effects

### Opacity (Coverage of the Film)

The positive values indicate a decrease in the visibility of the parameter, whereas the negative values indicate an accentuation of the visibility of the parameter. By way of example, the negative values for the effect on the relief indicate an accentuation of the relief and the positive values indicate a smoothing out effect.

The results obtained are indicated in the following table:

| | | Smoothing | | |
|---|---|---|---|---|
| | Effects on | effects | “Colour” | Opacity |
| | wrinkles/fine | Dilated | corrector | (Coverage of |
| Composition | lines | pores | effects | the film) |
| Example 9 | 3 | 19 | 25 | 10 |
| (invention) | | | | |
| Example 10 | 0 | 4 | 20 | 12 |
| (outside the | | | | |
| invention) | | | | |
| Example 11 | −3 | 8 | 20 | 12 |
| (outside the | | | | |
| invention) | | | | |

Example 9 of the invention showed a better correction of the colour imperfections while at the same time having the same opacity as the two formulae with the pigments conventionally used in foundations. This production of uniformity without introducing opacity makes it possible to achieve a natural uniformity-producing result. The combination of the invention also made it possible to achieve dilated-pore smoothing out performance levels that were much greater than with the other two formulae 10 and 11.

1: A composition, comprising, in a physiologically acceptable medium:
a) at least one soft-focus filler, and
b) at least one composite pigment comprising at least one non-spherical aluminium oxide, at least one metal oxide and at least one surface-treatment agent.

2: The composition according to claim 1, wherein the soft-focus filler has a Haze greater than 60% and a transmission TH greater than 60%.

3: The composition according to claim 1, wherein the soft-focus filler has a refractive index at 25° C. of between 1.33 and 2.

4: The composition according to claim 1, wherein the composition is an emulsion.

5: The composition according to claim 1, wherein the soft-focus filler is selected from
polytetrafluoroethylene powders,
polyurethane powders,
carnauba microwaxes,
synthetic wax microwaxes,
silicone resin powders,
hollow hemispherical particles of silicone,
acrylic polymer powders,
polyethylene powders, comprising at least one ethylene/acrylic acid copolymer,
crosslinked elastomeric organopolysiloxane powders and crosslinked elastomeric organopolysiloxane powders coated with silicone resin,
starch powders,
polyamide powders,
silica powders,
silicates,
talc of number-average size less than or equal to 3 microns,
silica/TiO₂ composites,
barium sulfate particles,
boron nitride particles,
composite powers of talc/TiO₂/alumina/silica,
spherical cellulose beads, and
mixtures of the foregoing.

6: The composition according to claim 1, wherein the soft-focus filler is selected from
polytetrafluoroethylene powders,
polyurethane powders,
carnauba microwaxes,
synthetic wax microwaxes,
silicone resin powders,
hollow hemispherical particles of silicone,
expanded vinylidene/acrylonitrile/methylene methacrylate microspheres,
copolymers of ethylhexyl acrylate and of at least one monomer selected from acrylic acid, methacrylic acid and mixtures and esters thereof,
polyethylene powders, comprising at least one ethylene/acrylic acid copolymer,
crosslinked elastomeric organopolysiloxane powders and crosslinked elastomeric organopolysiloxane powders coated with silicone resin,
starch powders,
silica particles surface-treated with a mineral wax,
hydrophobic silica aerogel particles,
talc of number-average size less than or equal to 3 microns,
silica/TiO₂ composites,
barium sulfate particles,
composite powers of talc/TiO₂/alumina/silica,
spherical cellulose beads, and
mixtures of the foregoing.

7: The composition according to claim 1, comprising
a) at least one soft-focus filler, other than boron nitride,
b) at least one composite pigment comprising at least one non-spherical aluminium oxide, at least one metal oxide and at least one surface-treatment agent, and
c) boron nitride particles.

8: The composition according to claim 7, wherein the soft-focus filler is selected from
polytetrafluoroethylene powders,
polyurethane powders,
carnauba microwaxes,
synthetic wax microwaxes,
silicone resin powders,
hollow hemispherical particles of silicone,
acrylic polymer powders,
polyethylene powders, comprising at least one ethylene/acrylic acid copolymer,
crosslinked elastomeric organopolysiloxane powders and crosslinked elastomeric organopolysiloxane powders coated with silicone resin,
starch powders,
silica particles surface-treated with a mineral wax,
hydrophobic silica aerogel particles,
silicates,
talc of number-average size less than or equal to 3 microns,
silica/TiO₂ composites,
barium sulfate particles,
composite powers of talc/TiO₂/alumina/silica,
spherical cellulose beads, and
mixtures of the foregoing

9: The composition according to claim 1, wherein the soft focus filler is selected from
spherical powders of crosslinked polymethyl methacrylate polymer, and
crosslinked copolymers of at least one monomer selected from acrylic acid, methacrylic acid and mixtures and esters thereof.

10: The composition according to claim 1, wherein the aluminium oxide is in a planar particle or platelet form, having an average diameter ranging from 1 to 20 μm and a thickness of less than 0.5 μm.

11: The composition according to claim 1, wherein the surface-treatment agent is an alkylsilane.

12: The composition according to claim 1, wherein the surface-treatment agent is present in a proportion of at least 0.50% by weight relative to a total weight of the composite pigment.

13: The composition according to claim 1, wherein the surface-treatment agent is present in a proportion of less than 30% by weight relative to a total weight of the composite pigment.

14: The composition according to claim 1, wherein the non-spherical aluminium oxide is in platelet form and comprises an upper surface and a lower surface which are separated by a border and the metal oxide randomly covers said surfaces, but the border is substantially free of metal oxide.

15: The composition according to claim 1, wherein 5% to 99% of a surface of the non-spherical aluminium oxide is covered with the metal oxide of the composite pigment.

16: The composition according to claim 1, wherein the metal oxide is selected from titanium oxides and yellow, red, brown, amber or black iron oxides.

17: The composition according to claim 1, wherein the metal oxide is present in an amount ranging from 25% to 85% by weight relative to a total weight of the composite pigment.

18: The composition according to claim 1, wherein the composite pigment is selected from
(i) a pigment comprising at least one titanium oxide, a platelet alumina and an alkylsilane;
(ii) a pigment comprising an iron oxide, a platelet alumina and an alkylsilane; and
(iii) mixtures thereof.

19: The composition according to claim 7, wherein:
the soft-focus is present in a concentration ranging from 2% to 35% by weight relative to a total weight of said composition;
the composite pigment is present in a concentration ranging from 0.01% to 80% by weight relative to the total weight of the composition;
the boron nitride particles are present in a concentration ranging from 0.1% to 20% by weight relative to the total weight of the composition.

17-19. (canceled)

20: The composition according to claim 7, wherein the boron nitride particles are turbostratic.

21: The composition according to claim 7, wherein the boron nitride particles have an oxygen content ranging from 0.05% to 3% by weight, relative to a total weight of the particles.

22: The composition according to claim 7, wherein the boron nitride particles have an average particle size ranging from 0.1 to 25 μm.

23: A process for coating keratin materials, comprising applying to the keratin materials the composition according to claim 1.

24: The composition according to claim 1, wherein a weight ratio of the composite pigment/soft-focus filler ranges from 0.01 to 4.

25: The composition according to claim 1, comprising:
a) at least one soft-focus filler selected from acrylic polymer powders; and
b) at least one composite pigment comprising an iron oxide and/or a titanium oxide, a platelet alumina and a tri(C₁-C₄)alkoxy(C₁-C₁₂)alkylsilane.

26: The composition according to claim 7, comprising:
a) at least one soft-focus filler selected from acrylic polymer powders; and
b) at least one composite pigment comprising an iron oxide and/or a titanium oxide, a platelet alumina and a tri(C₁-C₄)alkoxy(C₁-C₁₂)alkylsilane.
